(19) United States
(12) Patent Application Publication
(10) Pub. No.: US 2004/0044368 A1
(43) Pub. Date: Mar. 4, 2004

(54) Sequential bipolar left-ventricle and right-ventricle pacing

(76) Inventors: Frits W. Prinzen, Maastricht (NL); Chester Struble, Eijsden (NL)

Correspondence Address: MEDTRONIC, INC., 710 MEDTRONIC PARKWAY NE, MS-LC340, MINNEAPOLIS, MN, 55432-5604, US

(21) Appl. No.: 10/422,710

(22) Filed: Apr. 24, 2003

(30) Foreign Application Priority Data
May 23, 2002 (NZ) 519153

(51) Int. Cl. A61N 1/00

(52) U.S. Cl. 607/2

(57) ABSTRACT

The left-ventricle and right-ventricle of the heart are pacing with alternating polarity pulses to conserve power and provide other benefits. The left ventricle is stimulated with a first polarity pulse delivered to a first electrode implanted in left-ventricle endocardial tissue. An interval is delayed after the first electrode has begun stimulating the left-ventricle. Polarity is switched from the first electrode and a second electrode. The right ventricle is stimulated with a second polarity pulse delivered to the second electrode implanted in right-ventricle endocardial tissue.

### RELATED APPLICATIONS

This is a continuation-in-part of U.S. Application No. 60/333,762, filed Nov. 29, 2001, that has been converted to a nonprovisional application.

### FIELD OF THE INVENTION

This disclosure relates implantable medical devices more particularly to cardiac ventricular pacing.

### BACKGROUND OF THE INVENTION

Heart failure is a lifelong condition that affects approximately 5 million people in the United States. Cardiac resynchronization therapy (CRT) is a treatment for heart failure that involves pacing the left-ventricle pacing or bi-ventricular pacing that can significantly improve cardiac hemodynamics for heart failure patients with left bundle branch block (LBBB) conduction disorders. Cardiac resynchronization therapy is more complex than traditional pacing requiring at least one additional lead and has creates greater power demands than traditional pacing.

For the foregoing reasons, what is needed is a cardiac resynchronization system that is less complex and is more energy efficient.

### BRIEF SUMMARY OF THE INVENTION

The left-ventricle and right-ventricle of the heart are pacing with alternating polarity pulses to conserve power and provide other benefits. The left ventricle is stimulated with a first polarity pulse delivered to a first electrode implanted in left-ventricle endocardial tissue. An interval is delayed after the first electrode has begun stimulating the left-ventricle. Polarity is switched from the first electrode and a second electrode. The right ventricle is stimulated with a second polarity pulse delivered to the second electrode implanted in right-ventricle endocardial tissue.

### BRIEF DESCRIPTION OF THE DRAWINGS

FIG. 1 shows an environmental view of an implantable cardiac device (ICD).

FIG. 2 shows a more detailed environmental view of an ICD.

FIG. 3 shows a simplified block diagram of an ICD embodiment.

FIG. 4 shows a schematic of a lead for septal endocardial pacing of the left and right-ventricles embodiment, a guiding catheter, and a puncture tool.

FIG. 5 shows a schematic of an implanted lead for septal endocardial pacing of the left and right-ventricles embodiment.

FIG. 6. shows an enlarged schematic an implanted lead for septal endocardial pacing of the left and right-ventricles embodiment.

FIG. 7 shows a schematic of a three electrode embodiment of an implanted lead for septal endocardial pacing of the left and right-ventricles.

FIG. 8 shows a flowchart of a method for sequential bipolar left-ventricle and right-ventricle pacing.

### DETAILED DESCRIPTION OF THE INVENTION

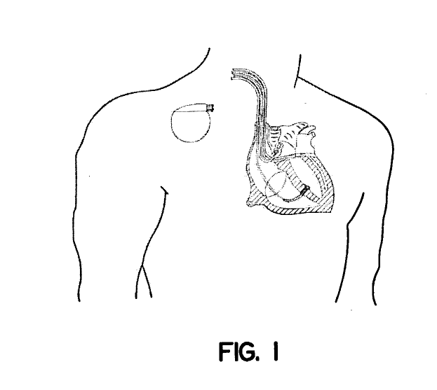

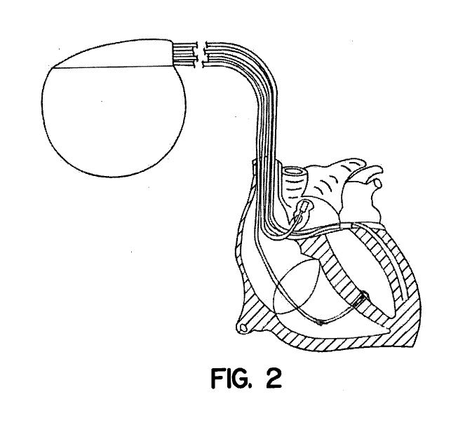

FIGS. 1 and 2 show an environmental view of an Implantable Cardiac Device (ICD) that can be used with a lead for septal endocardial pacing of the left and right-ventricles. The ICD can be any ICD capable pacing both the right-ventricle and the left-ventricle known as bi-ventricular pacing. Implantable Cardiac Devices suitable for bi-ventricular pacing include certain pacemakers, cardioverters, and defibrillators configured for bi-ventricular pacing. For example, the ICD and be an InSync® III Model 8040 pacemaker or an InSync® Marquis cardioverter/defibrillator available from Medtronic, Inc. in Minneapolis, Minn. USA.

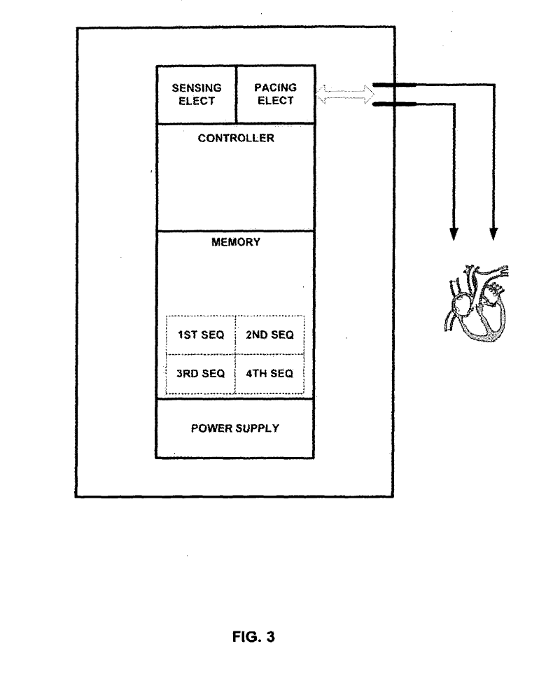

FIG. 3 shows a block diagram of an implantable cardiac device for optimal intra-left ventricular resynchronization. The cardiac pacemaker comprising a housing, a controller, memory, pacing electronics, sensing electronics, a first electrical lead, a second electrical lead, and software. The housing has a power supply carried in the housing and a feedthrough. The controller is carried in the housing and coupled to the power supply. Memory is coupled to the controller. The pacing electronics are coupled to the controller and the feedthrough. The sensing electronics coupled to the controller and the feedthrough.

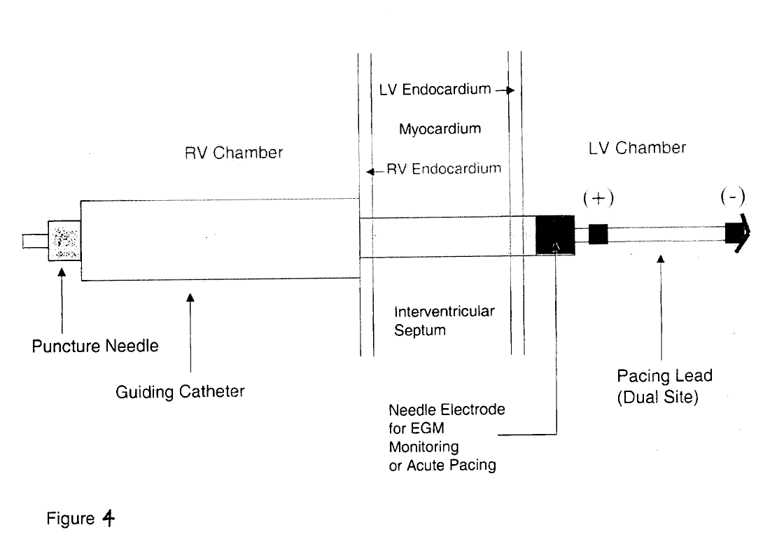

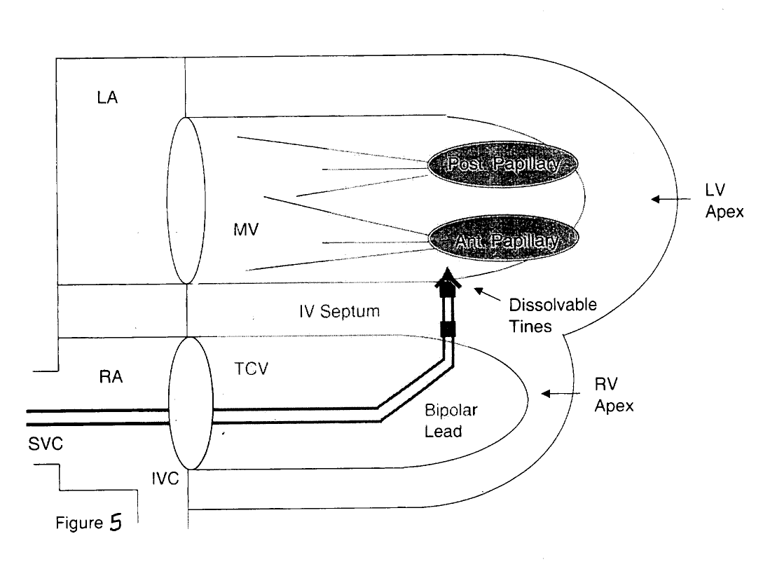

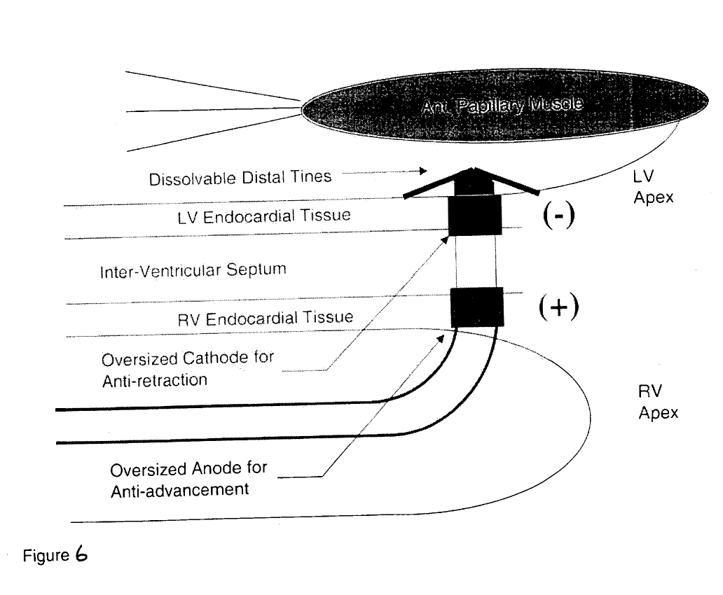

FIG. 4 shows a schematic of a lead for septal endocardial pacing of the left and right-ventricles embodiment, a guiding catheter, and a puncture tool. FIG. 5 shows a schematic of an implanted lead for septal endocardial pacing of the left and right-ventricles embodiment. FIG. 6. shows an enlarged schematic an implanted lead for septal endocardial pacing of the left and right-ventricles embodiment. The lead for septal endocardial pacing comprises a lead body, a first electrode, and a second electrode. The lead is a single lead that delivers electrical stimulation pacing to both the left-ventricle and the right-ventricle. The lead body has an inner lumen containing at least a first conductor and a second conductor. The lead body has a proximal end and a distal end. When the first electrode and second electrode are cathodes the implantable cardiac device can serve at an annode.

The lead for septal endocardial pacing comprises a lead body, a first electrode, and a second electrode. The lead is a single lead that delivers electrical stimulation pacing to both the left-ventricle and the right-ventricle. The lead body has an inner lumen containing at least a first conductor and a second conductor. The lead body has a proximal end and a distal end. When the first electrode and second electrode are cathodes the implantable cardiac device can serve at an annode.

The first electrode is carried on the lead body distal end and coupled to the first conductor. The first electrode is configured for placement in left-ventricle endocardial tissue. The first electrode is separated from the second electrode by less than about ______ mm. The first electrode's diameter can be greater than the lead body diameter and is typically at least ______ mm greater than the lead body diameter.

The second electrode is carried on the lead body distal to the first electrode and coupled to the second conductor. The second electrode is configured for placement in right-ventricle endocardial tissue. The first electrode is separated from the second electrode by less than about ______ mm. The second electrode's diameter can be greater than the lead body diameter and is typically at least ______ mm greater than the lead body diameter.

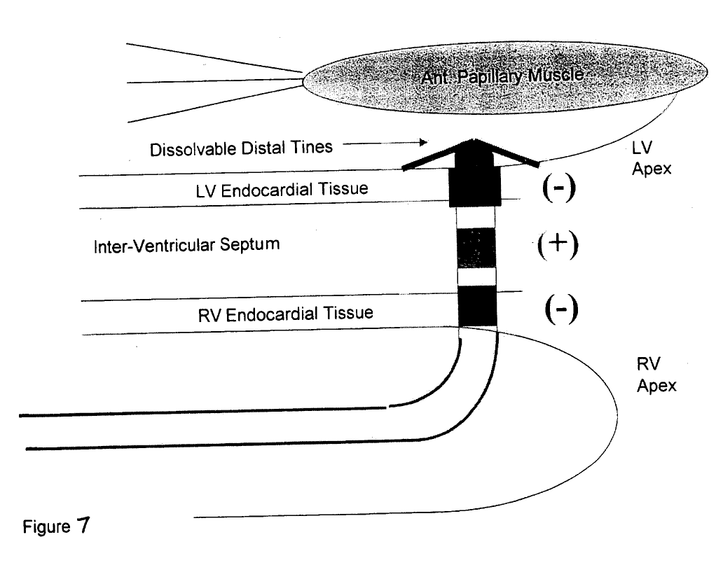

FIG. 7 shows a schematic of a three electrode embodiment of an implanted lead for septal endocardial pacing of the left and right-ventricles.

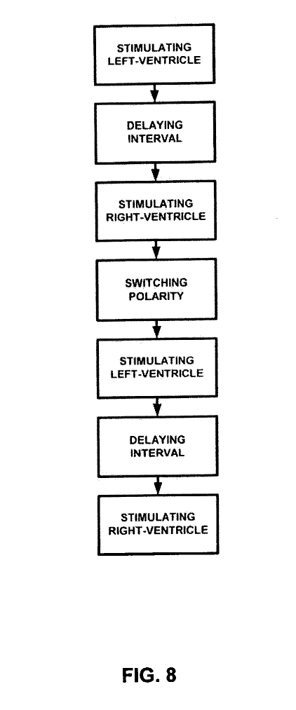

FIG. 8 shows a flowchart of a method for sequential bipolar left-ventricle and right-ventricle pacing embodiment. The method for sequential bipolar left-ventricle and right-ventricle pacing comprises stimulating a left ventricle, delaying an interval, stimulating a right-ventricle, switching polarity. The left ventricle is stimulated with a first polarity pulse delivered to a first electrode implanted in left-ventricle endocardial tissue. Stimulation is delayed for an interval after the first electrode has begun stimulating the left-ventricle. The interval can be in the range from about 0 ms to about 100 ms.

The right-ventricle is stimulated with a second polarity pulse delivered to the second electrode implanted in right-ventricle endocardial tissue. The first electrode and second electrode polarity is switched. The first electrode switches polarity to function alternatively as a first cathode and a first anode and the second electrode switches polarity to function alternatively as a second cathode and a second anode. When the first electrode receives the first polarity pulse, the first electrode functions as a cathode. When the second electrode receives the second polarity pulse, the second electrode functions as an anode. When either the first electrode or the second electrode function as an annode, the anode typically receives a higher stimulation signal than the cathode. The left-ventricle is stimulated with a second polarity pulse delivered to the first electrode implanted in left-ventricular endocardial tissue. Some embodiments of the bi-polar dual site pacing use only the first electrode and the second electrode for pacing. Energy consumption is reduced because the first polarity and the opposite polarity serve to reduce the need for charge balancing. Embodiment of the method for sequential bipolar left-ventricle and right-ventricle pacing can be performed as sequences of instructions that are executed by the controller.

Thus, embodiments of sequential bipolar left-ventricle and right-ventricle pacing are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method for sequential bipolar left-ventricle and right-ventricle pacing, comprising:
stimulating a left ventricle with a first polarity pulse delivered to a first electrode implanted in left-ventricle endocardial tissue;
delaying an interval after the first electrode has begun stimulating the left-ventricle;
stimulating a right-ventricle with a second polarity pulse delivered to the second electrode implanted in right-ventricle endocardial tissue;
switching polarity of the first electrode and a second electrode; and,
stimulating a left ventricle with a second polarity pulse delivered to a first electrode implanted in left-ventricle endocardial tissue.

2. The method as in claim 1 wherein bi-polar dual site pacing is accomplished with only the first electrode and the second electrode.

3. The method as in claim 1 wherein the interval is in the range from about 0 ms to about 100 ms.

4. The method as in claim 1 wherein first electrode switches polarity to function alternatively as a first cathode and a first anode and the second electrode switches polarity to function alternatively as a second cathode and a second anode.

5. The method as in claim 1 wherein the first electrode receiving the first polarity pulse functions as a cathode and the second electrode receiving the second polarity pulse functions as an anode.

6. The method as in claim 1 wherein the anode receive a higher stimulation signal than the cathode.

7. The method as in claim 1 wherein energy consumption is reduced because the first polarity and the opposite polarity serve to reduce the need for charge balancing.

8. The method as in claim 1 further comprising,
delaying an interval after second electrode has stimulated the right-ventricle; and,
stimulating a right-ventricle with a first polarity pulse delivered to the second electrode implanted in right-ventricle endocardial tissue.

9. A method for sequential bipolar left-ventricle and right-ventricle pacing, comprising:
means for stimulating a left ventricle with a first polarity pulse delivered to a first electrode implanted in left-ventricle endocardial tissue;
means for delaying an interval after the first electrode has begun stimulating the left-ventricle;
means for switching polarity of the first electrode and a second electrode; and,
means for stimulating a right-ventricle with a second polarity pulse delivered to the second electrode implanted in right-ventricle endocardial tissue.

10. The method as in claim 1 further comprising,
means for delaying an interval after second electrode has stimulated the right-ventricle;
means for stimulating a left ventricle with a second polarity pulse delivered to a first electrode implanted in left-ventricle endocardial tissue;
means for delaying an interval after the first electrode has begun stimulating the left-ventricle;
means for stimulating a right-ventricle with a first polarity pulse delivered to the second electrode implanted in right-ventricle endocardial tissue.

11. A cardiac pacemaker for sequential bipolar left-ventricle and right-ventricle pacing, comprising:
a housing having a power supply carried in the housing and a feedthrough;
a controller carried in the housing coupled to the power supply;
memory coupled to the controller;
pacing electronics coupled to the controller and the feedthrough;
sensing electronics coupled to the controller and the feedthrough;
a lead coupled to the feedthrough and configured for positioning in the right ventricle;
a first electrode carried on the lead body distal end and coupled to the first conductor, the first electrode is configured for placement in left-ventricle endocardial tissue;
a second electrode carried on the lead body distal to the first electrode and coupled to the second conductor, the second electrode is configured for placement in right-ventricle endocardial tissue; and,
software stored in memory containing instructions including,
a first sequence of instructions when executed by the controller, causes the controller to initiate stimulation of a left ventricle with a first polarity pulse delivered to a first electrode implanted in left-ventricle endocardial tissue,
a second sequence of instruction when executed by the controller, causes the controller to delay an interval after the first electrode has begun stimulating the left-ventricle,
a third sequence of instruction when executed by the controller, causes the controller to switch polarity of the first electrode and a second electrode, and,
a forth sequence of instruction when executed by the controller, causes the controller to initiate stimulation of a right-ventricle with a second polarity pulse delivered to the second electrode implanted in right-ventricle endocardial tissue.

12. The method as in claim 11 wherein bi-polar dual site pacing is accomplished with only the first electrode and the second electrode.

13. The method as in claim 11 wherein the interval is in the range from about 0 ms to about 100 ms.

14. The method as in claim 11 wherein first electrode switches polarity to function alternatively as a first cathode and a first anode and the second electrode switches polarity to function alternatively as a second cathode and a second anode.

15. The method as in claim 11 wherein the first electrode receiving the first polarity pulse functions as a cathode and the second electrode receiving the second polarity pulse functions as an anode.

16. The method as in claim 11 wherein the anode receive a higher stimulation signal than the cathode.

17. The method as in claim 11 wherein energy consumption is reduced because the first polarity and the opposite polarity serve to reduce the need for charge balancing.
